(19) United States
(12) Patent Application Publication
(10) Pub. No.: US 2007/0157400 A1
(43) Pub. Date: Jul. 12, 2007

(54) METHOD, APPARATUS AND KIT OF APPARATUS FOR COSMETICALLY TREATING AREAS OF HAIR LOSS

(76) Inventor: Tracy Ali, Detroit, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/616,429

(22) Filed: Dec. 27, 2006

Related U.S. Application Data
(60) Provisional application No. 60/754,121, filed on Dec. 27, 2005.

(51) Int. Cl. A61K 8/02

(52) U.S. Cl. 8/406; 424/401

(57) ABSTRACT

In order to color areas of scalp exposed when a woman's hair is styled so as to reveal areas of the scalp within the hairstyle, the colored areas are first treated with a light coating of hair gel which is allowed to dry, then are colored using a cosmetic pencil having a core of pigments supported in a polymer emulsion surrounded by a sheath that may be partially removed to expose extended sections of the core. The colored sections are then lightly sprayed with a spritz. The implements used to practice the method are preferably provided in a kit formed within an openable pouch, container, or the like, so that the implements are conveniently available and may be carried to touch up the hair after wear.

### FIELD OF THE INVENTION

This invention relates to a method of applying a cosmetic to scalp areas which are devoid of hair, to an apparatus for practicing the method, and to a kit comprising the elements of the apparatus.

### BACKGROUND OF THE INVENTION

Several factors produce hair loss in females along the sides of the head. One of the factors is the practice of dressing children's hair in tight rear knots or ponytails that pull the hairs along the sides of the scalp in the rear direction. Another factor is the application of straightening solutions to the hair which weaken the roots. In black women, in particular, both of these practices produce substantial thinning of the hair at the sides of the temple so that areas of the scalp are visible when hair is pulled back into a ponytail or the like. Alopecia may also produce hair loss. As a result, when women affected by these conditions wear their hair in styles which tend to group the hairs such as ponytails or sharp parts, areas of scalp are often visible.

It has been proposed to provide sprays of coloring material to mask these exposed scalp areas. For example, see U.S. Pat. No. 6,814,957. However, sprays of course cover the hair area as well as the scalp area and tend to accumulate in the hair, providing a glossy appearance and requiring frequent shampooing.

### SUMMARY OF THE INVENTION

The present invention is accordingly directed to a method of treating balding areas to color them like the hair and thus make them invisible, which does not require spraying, to a cosmetic applicator useful in the practice of the method, and to a kit of those implements useful in practicing the method.

Broadly, the method of the present invention involves use of a cosmetic applicator in the form of a pencil having an elongated core of the cosmetic bound together in a sufficiently rigid manner that it will not easily crumble when pressed against the scalp but yet sufficiently loose to allow the coloring material to coat the scalp areas to which it is applied. This core is supported in an outer cover which may be removed as the cosmetic core wears as a result of use. The cover may take the form of a wooden sheath of the type employed in a lead pencil or a spirally wound strip of material which may be plastic or cellulose based and may be partially unwound to reveal a fresh section of the cosmetic core after the core has become worn as a result of use.

The cover performs the function of supporting the fragile cosmetic core during the pressures exerted when the cosmetic is applied to the scalp and also of shielding the hand of the user from the cosmetic.

The cosmetic core itself may be made available in a range of colors to match the hair color of different users.

The cosmetic preparation itself is preferably water soluble so that it may be easily removed during washing or shampooing of the hair. It preferably forms a continuous, opaque coating in the areas in which the pencil is used to bring the core into pressured contact with the scalp. The cosmetic may be formulated in accordance with any known conventional manner of the type used with mascara, eye shadow sticks, eyeliner pencils or eyebrow pencils. The preferred composition of the cosmetic core constitutes a pigment of the desired color dispersed in some form of polymer such as acrylate or methacrylate. For example, the compositions and methods of forming those compositions disclosed in U.S. Pat. No. 3,697,643 would be suitable methods for forming the cosmetic cores of the applicator pencils of the present invention.

The method of the present invention is particularly applicable to allow women of color to wear their hair in forms which draw hairs from the front of the head, and particularly the temples, in a rearward direction. Because of the hair losses associated with childhood use of hair straightening compounds and hairstyles which draw the hairs to the rear, or alopecia, these women often suffer from thinning hair on the sides of the temple. When they draw their hair back in these common styles, the thinning areas may be highly visible. A method of the present invention involves first applying a hair gel to the hair and drawing the hair back into a bun, ponytail, sharp part or the like. Next, hair gel is applied to the visible scalp areas. This may be done by placing some hair gel on the fingers and rubbing it into the visible bald areas to form a thin coating. Next, a cosmetic pencil formed in accordance with the present invention and having a color matching the user's hair color is used to apply cosmetic to the balding areas between the hair strands. This may be done by holding the pencil at an angle to the scalp and filling in the bald areas. The cosmetic coating must be allowed to dry, either by rubbing, air drying or applying a flow of warm air with a hairdryer. Next, a very light coating of non-greasy hairspray or spritz for fixing the hair is sprayed over the coated area. The hairdo which results has the appearance of being fall and contiguous and is free of the negative effects of the use of cosmetics and spritz.

### BRIEF DESCRIPTION OF THE DRAWINGS

Other objects, advantages and applications of the present invention will be made apparent by the following detailed description of the preferred embodiment of the invention. The description makes reference to the accompanying drawings in which:

FIG. 1 is a side view of a woman's head with her hair drawn back in a knot and illustrating streaks in which the scalp is visible as a result of thinning of the hair;

FIG. 2 is a top view of a woman's head with her hair parted in the center to reveal a balding area;

FIG. 3 is a perspective view of a cosmetic pencil formed in accordance with the present invention for use in coloring the exfoliated areas of the type illustrated in FIGS. 1 and 2;

FIG. 4 is a perspective view of a second form of pencil covered by spiral strips which may be removed to expose the cosmetic core as it wears during use; and

FIG. 5 illustrates a pouch in open form for containing the implements for practice of the present invention.

### DETAILED DESCRIPTION OF THE INVENTION

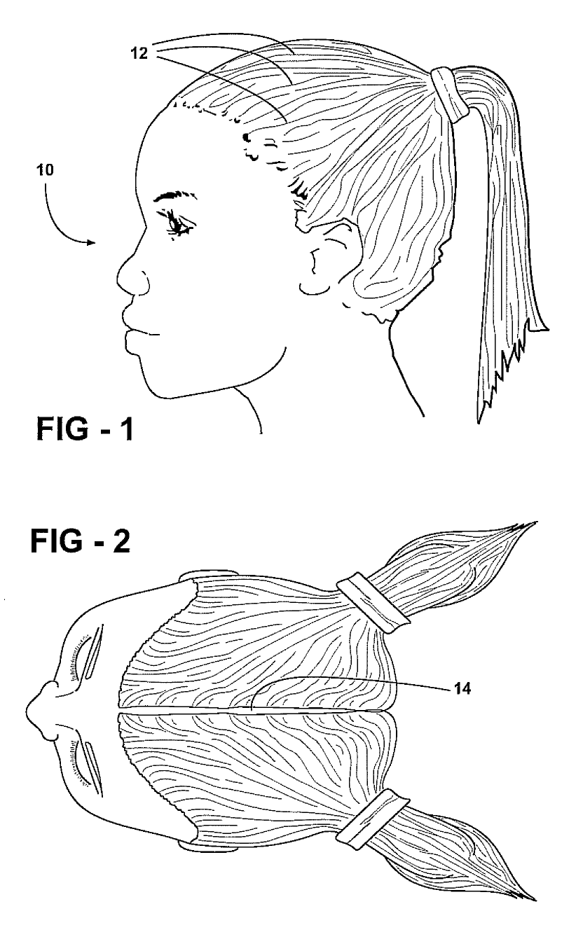

Referring to FIG. 1, a woman's head, generally indicated at 10, is illustrated with her hair drawn back into a bun, ponytail or the like, so that the strands are pulled generally horizontally and to the rear of the head. Where thinning has occurred to the hair, for a variety of reasons, this produces streaks 12 toward the forward end of the temples, where the scalp is visible by virtue of thinning so that the relatively few hairs that are left gather in strands separated by the streaks 12.

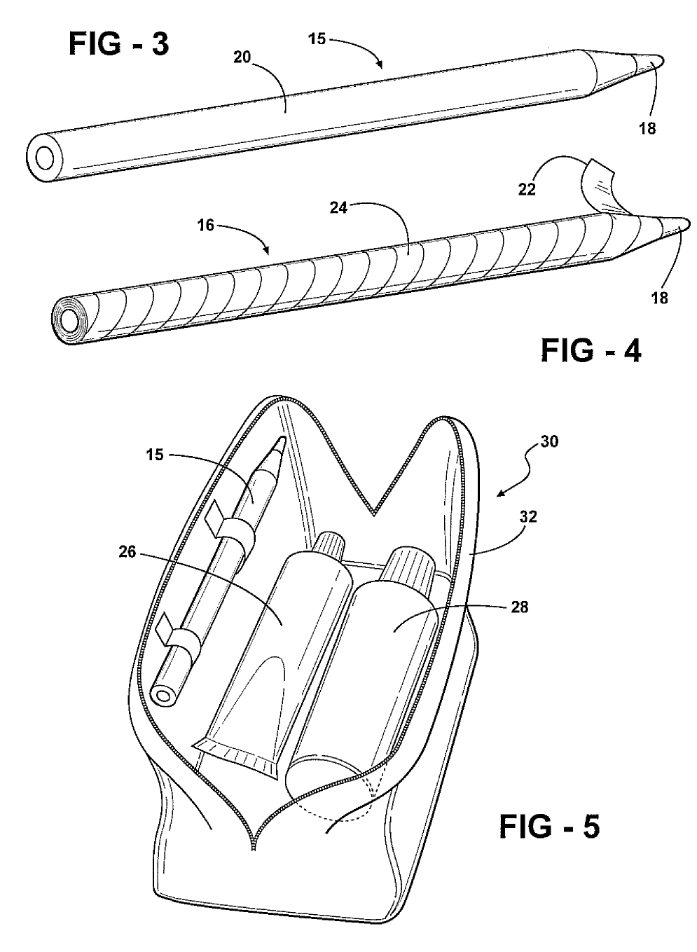

Similarly, FIG. 2 illustrates a hairstyle parted in the center revealing a balding area 14. In accordance with the present invention, these visible areas 12 and 14 may be colored to a similar color as the hair to mask the otherwise visible scalp areas and give the appearance of a full head of hair. This is preferably accomplished by using a pencil, of the type generally indicated at 15 in FIG. 3, or generally indicated at 16 in FIG. 4, These pencils both have a relatively small diameter, elongated, central core 18 of a coloring cosmetic composition. In the pencil 14 the core 18 is encompassed in a wooden sheath 20. As the tip of the core 18 wears as a result of being brought into pressure contact with the scalp areas 12, the extending tip may be removed by cutting away the forward end of the wood sheath 20 using a knife, pencil sharpener or the like.

The pencil 16 is covered by an elongated strip of plastic, cellulose, or other resilient material 22 which is spirally wound around the core 18 at 24. When the tip of the core is worn down, it may be removed by removing a length of the strip 22.

In the preferred embodiment of the invention the core 18 may be formed by an emulsion of suitable pigments, such as iron oxide, red iron oxide and the like, all of which are well known for use in cosmetic preparations, and a copolymer of an acrylate or methacrylate. A core formed in the manner of Synthetic Example 2 as disclosed in U.S. Pat. No. 4,423,031 is suitable for the formation of the present core. The composition, when brought into pressure and contact with the skin, produces a continuous film which is flexible yet hard and has excellent adhesive properties to the scalp. Moreover this film is generally water resistant yet may be easily removed by shampooing or the like.

In the practice of the method of the present invention, the hair is first styled in a manner such as is illustrated in FIG. 1 or 2, to reveal certain balding areas. This may be done with the hair slightly damp or lightly gelled, to allow the hair to lay in the preferred style. The exposed areas such as 12 or 14, which may result from balding, are then thinly coated with a hair gel. The hair gel may be any conventional variety marketed for that purpose, such as glycerin, mineral oil, lanolin, and may contain various additives such as pH adjusters, or anti-microbial preservatives such as ascorbic acid or the like. The gel is typically marketed in a dispensing tube, the type illustrated at 26 in FIG. 5, but also may be made available in other containers such as a jar. A small quantity of the gel is applied to the fingers of the person performing the method of the present invention and is rubbed into the visible balding areas 12, 14 or the like, to apply a thin coating fully covering the balding areas. The gel coating must be allowed to dry, which can be achieved by further rubbing, which thins the applied layer and exposes it to air-drying, or by applying warm air from a hairdryer, or simply waiting for the layer to dry out.

Next, the second step of the process, the tip 18 of the coloring pencil 15 or 16 is applied to the coated balding area so as to impart the color of the core of the pencil to the area. The color, of course, is chosen to match the color of the woman's hair.

Next, the third step of the process, the spritz coating is applied over the colored balding areas preferably using a spray can such as 28 in FIG. 5. In the course of this process, strays hair may be straightened into the preferred style.

When properly performed, the resulting head of hair appears full and continuous, without any visible balding areas, depending, as in any similar process, on the skill of the person performing the process.

The coloring in the balding areas may require touch-up after long use or as a result of being disturbed, such as by the user applying clothing which touches the head. The instruments for the practice of the process are accordingly preferably provided as a kit consisting of a pouch generally indicated at 30 which may have a separable closure, such as a zipper 32 to allow it to be opened or closed. Implements in the kit comprise a tube of hair gel, such as 26; a coloring pencil 15 of the type at FIG. 3 or 4, and a can of pressurized spritz 28.

To allow the user to conveniently practice the present invention and to touch up the resulting hairdo when the hairdo is disturbed as a result of sleeping on it or the like, a kit may be provided comprising a container either rigid or flexible which may contain a tube of hair gel, such as 26, a coloring pencil 15 formed in accordance with the invention and a can of spritz 28.

Other compositions including other pigments and bases known to the art can be used in connection with the present invention.

Having thus described my invention, I claim:

1. The method of cosmetically coloring areas of scalp visible after hair is styled, comprising:
drawing the hair into the preferred hairdressing form to reveal the scalp area; and
bringing the core end of an elongated pencil having a core of cosmetic surrounded by a sheath that may be partially removed to expose extended sections of the core against the balding areas to color the exposed sections.

2. The method of claim 1 wherein the core comprises pigments supported in a polymer emulsion.

3. The method of claim 1 further comprising the step of applying a thin layer of a hair gel to the exposed scalp areas, by manually rubbing the gel into the exposed areas and allowing it to dry, following the step of drawing the hair into the preferred hairdressing form, and prior to bringing the core end of an elongated pencil against the exposed sections of the scalp so as to color the exposed sections.

4. A cosmetic implement for coloring areas of scalp visible when hair is styled in the manner such as to expose areas of scalp within the hairstyle comprising an elongated pencil having a core comprising pigments supported in a polymer emulsion surrounded by a sheath that may be partially removed to expose extended sections of the core so as to allow the exposed sections of the scalp to be colored.

5. The cosmetic implement of claim 4 wherein the sheath comprises a spiral wound strip, one ended which may be removed to expose sections of the core.

6. A kit for cosmetically coloring areas of scalp visible when hair is styled so as to expose scalp areas, comprising an openable container supporting a tube of hair gel, a coloring pencil having a core comprising pigments supported in a polymer emulsion surrounded by a sheath that may be partially removed to expose extended sections of the core, and a can of spritz, whereby the components may be sequentially used to impart and retain color matching the hair in the exposed scalp areas.
